(19) United States
(12) Patent Application Publication
(10) Pub. No.: US 2004/0073132 A1
(43) Pub. Date: Apr. 15, 2004

(54) Systems and methods for detecting vulnerable plaque

(76) Inventors: Tracy Maahs, Rancho Santa Margarita, CA (US); Jesus Flores, Perris, CA (US); Thomas H. Campbell, Redwood City, CA (US); Duane Dickens, San Clemente, CA (US)

Correspondence Address: STRADLING YOCCO CARLSON & RAUTH, SUITE 1600, 660 NEWPORT CENTER DRIVE, P.O. BOX 7680, NEWPORT BEACH, CA, 92660, US

(21) Appl. No.: 10/431,671

(22) Filed: May 7, 2003

Related U.S. Application Data
(60) Provisional application No. 60/379,437, filed on May 7, 2002.
(60) Provisional application No. 60/412,359, filed on Sep. 20, 2002.

(51) Int. Cl. A61B 5/00

(52) U.S. Cl. 600/549

(57) ABSTRACT

A thermography system that can be used in the vasculature of a patient, or elsewhere, with a catheter and an instrument configured to graphically display thermography data from the catheter. Sensors on the catheter may measure blood temperature, wall temperature of a body vessel or both as well as differences between blood temperature and wall temperature. Temperature differences from different locations of a patient's body vessel can be measured contemporaneously, compared and displayed on the graphical display which may be a graphic user interface in some embodiments.

### RELATED APPLICATIONS

This application claims priority from U.S. Provisional Patent Application Ser. No. 60/379,437, filed May 7, 2002, titled Method and system for Treating Vulnerable Vascular Plaque, and U.S. Provisional Patent Application Ser. No. 60/412,359, filed Sep. 20, 2002, titled Real Time Thermography Catheter, both of which are incorporated by reference herein in their entirety.

### BACKGROUND

Coronary Artery Disease (CAD) is a leading cause of death in nearly all developed countries. For example, in the United States the National Institutes for Health estimates that some form of CAD afflicts nearly 7 million Americans and that CAD is a primary cause of death in over 500,000 persons annually. Coronary artery disease is defined as a reduction of blood flow to the heart as a result of an occlusion in a coronary artery. Reduced blood flow to the heart, or ischemia, may be asymptomatic, chronic or acute. Over time, many asymptomatic persons develop chronic CAD beginning with mild chest pain (angina) during or immediately following periods of physical exertion which may eventually lead to debilitating ischemia and persistent acute angina. However, in many cases asymptomatic CAD can develop into acute coronary syndromes including unstable angina, myocardial infarction (MI) and even sudden death.

Both chronic and acute CAD result from atherosclerotic plaques formed on the artery's intimal layer (the innermost lining of the blood vessel composed of endothelial cells) in response to an injury. (P. K. Shah. 1997. Plaque Disruption and Coronary Thrombosis: New Insight into Pathogenesis and Prevention. Clin. Card. Vol. 20 (Suppl. II), II-38-II-44.) A variety of atherosclerotic plaques are known to exist. Moreover, the type of atherosclerotic plaque formed within the blood vessel dictates whether the resulting CAD will be a stable chronic condition or acute CAD possibly resulting in sudden death. (Id.) Atherosclerotic plaques are generally composed of a fibrous outer layer, or cap, and soft atheromatous core of fatty material referred to as atheromatous gruel. The exact composition of mature atherosclerotic plaques varies considerably and the factors that effect an atherosclerotic plaque's make-up are poorly understood. However, the fibrous cap associated with many atherosclerotic plaques is formed from a connective tissue matrix of smooth muscle cells, types I and III collagen, and a single layer of endothelial cells. The atheromatous gruel is composed of blood-borne lipoproteins trapped in the sub-endothelial extracellular space and the breakdown of tissue macrophages filled with low density lipids (LDL) scavenged from the circulating blood. (G. Pasterkamp and E. Falk. 2000. Atherosclerotic Plaque Rupture: An Overview. J. Clin. Basic Cardiol. 3:81-86). The ratio of fibrous cap material to atheromatous gruel determines plaque stability and type.

There are two predominate populations of atherosclerotic plaques (Id). The plaque associated with stable chronic CAD is commonly referred to as fibrointimal lesions which are composed of fibrous tissue with minimal, if any, atheromatous gruel. Fibrointimal plaques are generally quite stable and are associated with gradual luminal narrowing eventually leading to myocardial ischemia and angina. These plagues are composed of 70% or more hard, collagen-rich sclerotic tissues and are less likely to rupture. Consequently, survival rates associated with this type of plaque are generally good and the resulting ischemic heart disease is treated with vasodilators, angioplasty, and angioplasty with stenting or coronary bypass graft surgery. However, when a thick hard sclerotic cap does not support the atheromatous gruel rich core, the plague is subject to rupture. This type of plaque is referred to as vulnerable plaque and poses a great threat for acute CAD and sudden death (Id). The unstable atherosclerotic plaque associated with acute CAD including unstable angina, myocardial infarction (MI) and even sudden death are comprised of lipid-laden lesions having a soft central core and a thin fibrous cap (Id).

Atherosclerotic plaque forms in response to vascular endothelial cell injury associated with, among other causes, hyper-cholesterolemia, mechanical trauma, and autoimmune diseases. The injured endothelial cells secrete chemotactic and growth factors such as monocyte chemotactic protein 1 that cause circulating monocytes to converge on the injured site and attached to the endothelium. The monocytes then migrate into the sub-endothelium where they undergo a phenotypic transformation into tissue macrophages. The tissue macrophages may scavenge LDL present in the blood stream and may ultimately form foam cells and fatty streaks that eventually mature into atherosclerotic plaque (M. Navab, et al. 1991. Monocyte Transmission Induced by Modification of LDL in Co-culture of Human Aortic Wall Cells is Due to Induction of Monocyte Chemotactic Protein I Synthesis and Abolished by HDL. J. Clin. Invest. 88:20392040).

The vulnerability of plaque may be determined by examining a combination of intrinsic properties and extrinsic factors. For example, the three most important intrinsic factors that predispose plaques to rupture include the characteristics of the atheromatous core, the characteristics of the fibrous cap, and cap fatigue and inflammation.

The first intrinsic factor affecting plaque vulnerability pertains to the characteristics of the atheromatous core. Atherosclerotic plaque begins to become increasing more unstable, and hence more vulnerable to rupture, when the lipid-laden core exceeds 40% of the total structure (B. Lundberg. 1985. Chemical Composition and Physical State of Lipid Deposits in Atherosclerosis. Atherosclerosis, 56:93-110). Furthermore, core composition is important in determining plaque vulnerability. Atherosclerotic gruel having increased amounts of extracellular lipids in the form of cholesterol esters (as opposed to cholesterol crystals) is particularly soft and increases plaque vulnerability. Moreover, inflammation and infection raise body temperature causing the plaque's cholesterol ester-rich gruel core temperature to increase. As the core warms it becomes increasingly unstable and susceptible to rupture.

The second intrinsic factor affecting plaque vulnerability is directed to the characteristics of the fibrous cap, and more particularly to the cap thickness and content. Cap cellularity, matrix composition and collagen content varies considerably (M. J. Davis, et al. 1993. Risk of Thrombosis in Human Atherosclerotic Plaques: Role of Extracellular Lipid, Macrophages and Smooth Muscle Cell Content. Br. Heart J. 69:377-381). Generally, caps having fewer collagen synthesizing cells are inherently weaker than caps with higher collagen content. Therefore, the collagen content determines a cap's tensile strength, particularly at the junction between the plaque and adjacent vessel wall. This region, referred to as the plaque shoulder, is often the thinnest portion of the cap and may be heavily infiltrated with macrophages and foam cells. Consequently, the plaque shoulder region is inherently unstable the site were rupture usually occurs.

The third intrinsic factor affecting plaque vulnerability pertains to cap fatigue and inflammation. Cap inflammation has been identified as a potential factor in plaque rupture leading to acute coronary syndromes (E. Falk, et al. 1995. Coronary Plaque Disruption. Circulation, 92:657-671). Disrupted fibrous caps taken post mortum from patients with unstable angina are often more heavily infiltrated with macrophages at the plaque rupture site than plaque from cases of stable angina. In addition to Macrophages, other cells involved in the inflammatory response are also found in atherosclerotic plaque. T lymphocytes, mast cells and neutrophils secrete cytokine and protolytic enzymes that contribute to plaque instability. Activated T-cells infiltrate the plaque and compromise plaque structural integrity by secreting interferon-γ (INF-γ) which in turn down regulates collagen synthesis within the fibrous cap, inhibits vascular smooth muscle cell (VSMC) proliferation and induces VSMC apoptosis. Furthermore, INF-γ also activates tissue macrophages present in the lesion as well as circulating macrophages (P. R. Moreno, et al. 1996. Macrophages, Smooth Muscle Cells, and Tissue Factor in Unstable Angina. Implications for Cell-Mediated Thrombogenicity in Acute Coronary Syndromes. Circulation. 94: 3090-3097).

Activated macrophages secrete protolytic proteins that degrade the caps extracellular matrix decreasing cap thickness as well as increasing macrophage infiltration which contributes to gruel mass and shoulder instability.

Recently, a group of proteolytic enzymes known as matrix metalloproteinases have been shown to attack and degrade the fibrillar interstitial collagen characteristic of plaque caps. (G. K. Sukhova, et al. 1999. Evidence for Increased Collagenolysis by Interstitial Collagenases-1 and -3 in Vulnerable Human Atheromatous Plaques. Circulation; 99:2503-2509; see also Z. Galis, et al. 1994. Increased Expression of Matrix Metalloproteinases and Matrix Degrading Activity in Vulnerable Regions of Human Atherosclerotic Plaques. J. Clin. Invest.; 94: 2493-2503; see also C. M. Dollery, et al. 1995. Matrix Metalloproteinases and Cardiovascular Diseases. Circ. Res.; 77:863-868).

Like the aforementioned intrinsic properties, a number of extrinsic factors may trigger a rupture of a vulnerable atherosclerotic plaque. These extrinsic factors include the physical stresses endured by the arterial wall such as circumferential forces, compressive forces, circumferential bending, longitudinal flexion and hemodynamic forces. Circumferential forces within a vessel lumen are determined by blood volume, blood pressure and lumen diameter. The circumferential pressure increases as blood volume and pressure increase. The narrower the vessel lumen, the greater the circumferential pressure will be for any given blood volume or pressure. Circumferential forces exert pressure against the vessel wall which is resisted by the circumferential tension. Without circumferential tension, the vessel wall would continue to expand until aneurysm results. However, the circumferential tension is not exerted by the vessel wall exclusively, vessel wall structures such as plague also exert tension in response to the circumferential forces (A. Maclssac, et al. 1993. Toward the Quiesent Coronary Plaque. J. Am. Coll. Cardiolo., 22:1228-1241).

Plaques associated with stable CAD have thick fibrous caps and minimal soft atheromatous core. Consequently, as circumferential force increases within the vessel the resulting circumferential tension is distributed throughout the thick fibrous cap with minimal load bearing being done by the soft gruel. As a result the lesion remains stable and resists rupture. However, as the gruel content increases and cap thickness decreases, the circumferential tension cannot be adequately dissipated by the fibrous cap. As a result, increased pressure from the lumen is exerted on the soft atheromatous core. Once this pressure reaches a critical point the cap ruptures, usually at the shoulder region.

Fibrous cap compression is essentially the opposite of circumferential force. Circumferential force results from tension created as the vessel lumen resists expansion. The greater the pressure within the lumen, the greater the circumferential tension that must be applied to resist aneurysm. As the tension mounts within the lumen wall, it is communicated directly to the interior of attached structures such as plaque. Consequently, the greater the circumferenfal force, the greater the pressures become against the plaque core. As previously explained, plaques having a higher fibrous cap to soft atheromatous core ratio are better able to distribute the luminal pressure and resist rupturing. Plaque compression often results from vasospasm where the lumen wall presses against these structures compressing the plaque core. Plaques having a greater volume of soft atheromatous core and a thin fibrous cap are most prone to compression rupture (R. T. Lee and R. D. Kamm. 1994. Vascular Mechanics for the Cardiologist. J. Am. Coll. Cardiol. 23; 1289-1295).

Other extrinsic mechanical factors such as circumferential bending and longitudinal flexion are believed to be less important than cap tension and compression in plaque rupture. Circumferential bending is caused by the normal pulse wave generated within the vessel lumen associated with changes in luminal blood pressure. During the diastolic-systolic cycle the lumen diameter will change approximately 10 percent (Id). This constant fluctuation in lumen diameter results in circumferential bending of the atherosclerotic plaque. Longitudinal flexion results from the normal beating of the heart. Coronary arteries anchored to the myocardium are constantly stretched and relaxed as the heartbeats. This exerts a longitudinal stress on the vessel lumen which is directly communicated to attached structures such as atherosclerotic plaque. The combined actions of circumferential bending and longitudinal flexing exert forces on the plaque fibrous cap as described above. Thus, the thicker the cap the more resistant to rupture the plaque becomes (Id).

The hemodynamic factors are non-mechanical in nature and probably contribute the least to plaque rupture. Hemodynamic forces are generally associated with shear stress. Shear force result from turbulence created as a fluid change velocity in response to topological changes in the arterial wall (M. L. Armstrong, et al. 1985. Structural and Hemodynamic Responses to Peripheral Arteries of Macaque Monkeys to Atherosclerotic Diet. Arteriosclerosis. 5:336-346). For example, blood flowing through an artery having a fixed diameter moves at a constant speed. However, when the blood flow reaches a stricture in the vessel caused by plaque, it accelerates through the narrowing consistent with Bernoulli's principle. As the blood flow passes the narrowed lumen region it slows creating vortices in the blood flow that can theoretically disrupt the plaque. Obviously, stable plaques having thick caps will be less affected than plaques with thin caps and large volumes of atheromatous gruel.

Regardless of the cause, once plaque rupture occurs, thrombus formation is initiated. Rupture of the lipid-laden plaque exposes the highly thrombogenic atheromatous core and the sub-endothelium VSMC component of the arterial wall to the circulation. Platelet aggregation and adherence to the sub-endothelium follow this almost immediately. Platelet adhesion results in their activation and release of growth factors into the circulating blood and the initiation of the coagulation cascade. The released growth facts, specifically platelet-derived growth factor (PDGF) stimulates the proliferation and migration of VSMC. Proliferation and migration of VSMC can lead to plaque remodeling and increased vascular stenosis, or interact with the platelets leading to enhanced thrombogenesis (G. Pasterkamp and E. Falk. 2000. Atherosclerotic Plaque Rupture: An Overview. J. Clin. Basic Cardiol. 3:81-86).

The extent of vascular injury following plaque rupture determines the platelet adherence rates and thrombus formation. Platelet adherence and thrombus formation is complete within five to ten minutes when the injury to the vessel intima is superficial. The resulting thrombus is relatively unstable and is easily dislodged by blood flow shear forces. Once dislodged, the thrombus can be carried down stream causing unstable angina, MI or strokes (L. Badimon, et al. 1986. Influence of Arterial Wall Damage and Wall Sheer Rate on Platelet Deposition: Ex vivo Study in Swine Model. Arteriosclerosis. 6:312). Deep vessel injury results in enhanced platelet deposition and thrombus formation that is located deeper within the intimal or medial layers. These thrombi are less easily dislodged but can contribute to abrupt arterial occlusion and sudden death. However, regardless of the magnitude of vessel injury, once the coagulation cascade has been initiated, thrombi formed in the heart's vasculature present significant short and long term health risks (V. Fuster, et al. 1988. Insights into the Pathogenesis of Acute Ischemic Syndromes. Circulation. 77:1213-1220).

Stable plaques have minimal atheromatous gruel, thick caps, are relatively stable and generally do not present a risk of MI or sudden death. Stable plaques will most probably either result in progressive ischemic CAD or remain asymptomatic for life. However, as discussed above, vulnerable plaque can result in life threatening CAD including sudden death. Coronary artery disease associated with stable plaque can be effectively treated using minimally invasive procedures including angioplasty, stenting or medications. However, satisfactory acute therapies for treating vulnerable plaque are believed to be extremely limited.

Studies into the composition of vulnerable plaque suggest that the presence of inflammatory cells (and particularly a large lipid core with associated inflammatory cells) is the most powerful predictor of ulceration and/or imminent plaque rupture. For example, in plaque erosion, the endothelium beneath the thrombus is replaced by or interspersed with inflammatory cells. Recent literature has suggested that the presence of inflammatory cells within vulnerable plaque and thus the vulnerable plaque itself, might be identifiable by detecting heat associated with the metabolic activity of these inflammatory cells. Specifically, it is generally known that activated inflammatory cells have a heat signature that is slightly above that of connective tissue cells. Accordingly, it is believed that one way to detect whether specific plaque is vulnerable to rupture and/or ulceration is to measure the temperature of the plaque walls of arteries in the region of the plaque.

Once vulnerable plaque is identified, the expectation is that in many cases it may be treated. Therefore, it would be a significant advance in the treatment of CAD if methods were developed for treating vulnerable plaque coincident with detection. Since currently there is an ongoing need for devices to identify and locate vulnerable plaque, current treatments tend to be general in nature. For example, low cholesterol diets are often recommended to lower serum cholesterol (i.e. cholesterol in the blood). Other approaches utilize systemic anti-inflammatory drugs such as aspirin and non-steroidal drugs to reduce inflammation and thrombosis. However, it is believed that if vulnerable plaque can be reliably detected, localized treatments may be developed to specifically address the problems.

Thus, in light of the foregoing, there currently exists an ongoing need for systems and methods for identifying and treating vulnerable atherosclerotic plaque in vivo.

### SUMMARY

One embodiment is directed to a thermography system having a thermography catheter with a thermal sensor on a distal section thereof, a system controller coupled to the thermal sensor and a display configured to graphically display thermography data from the thermal sensor. The display may be a graphic user interface device. In one embodiment, the thermography catheter includes a plurality of thermal sensors in a substantially annular array and the display is configured to display thermography data from the sensors in a series of concentric rings each of which are divided into circumferential sections with each circumferential section correlating to a distinct thermal sensor and with each concentric ring representing a different thermography data point. In another embodiment, the thermography catheter includes a plurality of thermal sensors in a substantially annular array and wherein thermography data from the thermal sensors is displayed in an annular ring on a screen of the display that is divided into circumferential sections with each section corresponding to a thermal sensor. In yet another embodiment, a thermography instrument graphically displays thermography data from a thermography data input of the system controller on a graph having thermography data from the thermography data input on a first axis and an axial position of a site from which the thermography data was taken on a second axis. Thermography data displayed may include temperature data or temperature differential data from a thermal sensor or the like, including vessel wall temperatures or blood temperatures.

An embodiment of an apparatus for measuring thermal characteristics of a blood vessel in vivo includes a catheter having a proximal end, a distal end, and a distal section. An expandable slotted body is located at the distal section of the catheter and has one or more slotted body arms. One or more vessel wall temperature sensors are positioned on the slotted body arms and are configured to make contact with a vessel wall when the expandable slotted body is in an expanded state. One or more blood temperature sensors are positioned on the slotted body arms or an inner lumen of the apparatus in a configuration which prevents contact between the blood temperature sensors and the vessel wall when the expandable slotted body is in an expanded state.

A method of displaying thermography data includes providing a thermography system which includes a thermography catheter with a thermal sensor on a distal section thereof, a system controller coupled to the thermal sensor and a display configured to graphically display thermography data from the thermal sensor. The thermography catheter is positioned in a body of a patient and thermography data is detected at the thermal sensor. The thermography data is graphically displayed on the display. In some instances, the method is carried out with the thermal sensors positioned within a coronary artery of the patient. The display may be a graphical user interface in some embodiments.

In one embodiment, the thermography catheter includes a plurality of thermal sensors in a substantially annular array and the display graphically displays the thermography data in a series of concentric rings divided into circumferential sections with each circumferential section correlating to a distinct thermal sensor and with adjacent rings representing different thermography data points. In another embodiment, the thermography data is displayed in an annular ring on a screen of the display that is divided into circumferential sections.

### BRIEF DESCRIPTION OF THE DRAWINGS

FIG. 1 shows a block diagram of a thermography system;

FIG. 2 is a perspective view of an embodiment of a thermography instrument;

FIG. 3 shows another view of the embodiment of FIG. 2;

FIG. 4 shows an exploded view of an embodiment of an axial translation encoder;

FIG. 5 is a perspective view of an axial translation encoder having a catheter positioned thereon;

FIG. 6 shows a function actuator attached to a user interface;

FIG. 7 shows a “NEW” screen as displayed on a display module;

FIG. 7A shows an alternative embodiment of a “NEW” screen as displayed on a display module;

FIG. 8 shows a “SAVE” screen as displayed on a display module;

FIG. 9 shows an “OPEN” screen as displayed on a display module;

FIG. 10 shows a “CALIBRATE” screen as displayed on a display module;

FIG. 10A shows an alternative embodiment of a “CALIBRATE” screen as displayed on a display module;

FIG. 11 shows a “SETTINGS” screen as displayed on a display module;

FIG. 11A shows an alternative embodiment of a “SETTINGS” screen as displayed on a display module;

FIG. 12 shows a “SCAN” screen as displayed on a display module;

FIG. 13 shows an embodiment of a measurement screen as graphically displayed on a display module;

FIG. 14 shows another embodiment of a measurement screen as graphically displayed on a display module;

FIG. 15 shows another embodiment of a measurement screen as graphically displayed on a display module;

FIG. 16 shows another embodiment of a measurement screen with data displayed in concentric rings circumferentially segmented for each detector;

FIG. 16A shows another embodiment of a measurement screen with data displayed in concentric rings circumferentially segmented for each detector;

FIG. 17 shows a perspective view of an embodiment of a thermography catheter;

FIG. 18 shows a perspective view of an embodiment of a handle of a thermography catheter;

FIG. 19 shows a side perspective view of an embodiment of an elongated body of a thermography catheter;

FIG. 20 shows a cross-sectional view of an embodiment of an elongated body of a thermography catheter as taken along the lines 20-20 as shown in FIG. 19;

FIG. 21 shows an elevational view of an embodiment of an expandable slotted body of a thermography catheter in a non-deployed state;

FIG. 22 shows an elevational view of an embodiment of an expandable slotted body of a thermography catheter in a non-deployed state;

FIG. 23 shows view of an embodiment of an expandable slotted body of a thermography catheter in a deployed state within a vessel;

FIG. 24 shows an exploded view of an embodiment of a sensor and a slotted body arm of a thermography catheter;

FIG. 25 shows a perspective view of an embodiment of a sensor coupled to a slotted body arm of a thermography catheter;

FIG. 26 shows a perspective view of an embodiment of a sensor coupled to a slotted body arm of a thermography catheter;

FIG. 27 shows a cross sectional view of an embodiment of a sensor coupled to a slotted body arm of a thermography catheter as viewed along the line 27-27 as shown in FIG. 26;

FIG. 28 shows a cross-sectional view of an embodiment of a thermography catheter as taken along the lines 28-28 as shown in FIG. 22;

FIG. 29 shows a cross-sectional view of an embodiment of a thermography catheter as taken along the lines 29-29 as shown in FIG. 22; and

FIG. 30 shows a perspective view of a distal portion an embodiment of a thermography catheter in a deployed state.

### DETAILED DESCRIPTION

Disclosed herein is a detailed description of various illustrated embodiments of a thermography system. This description is not to be taken in a limiting sense, but is made merely for the purpose of illustrating general principles. The overall organization of the present detailed description is for the purpose of convenience only and is not intended to limit the present invention.

Systems and methods for identifying and treating vulnerable atherosclerotic plaque within a blood vessel of a patient are discussed herein. More particularly, systems for identifying and treating vulnerable plaque using various interventional devices are discussed. Exemplary interventional devices may include thermal mapping catheters and are intended to permit the diagnosis of body vessel regions that have a relatively higher heat production than comparable surrounding tissue and/or the temperature of adjacent luminal fluid (e.g. blood passing through a vessel, such as an artery, being mapped). In addition, thermal imaging capabilities may be combined with other therapeutic capabilities to provide integrated tools for diagnosis and/or treatment of specific conditions. For example, the present invention may be capable of delivering therapeutic agents to a localized area in vivo.

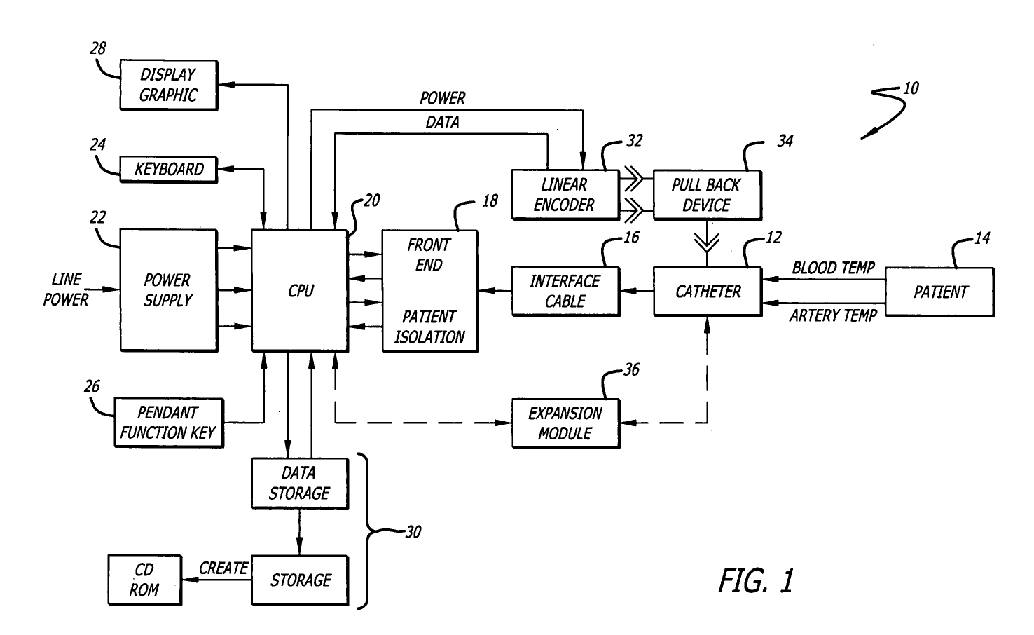

FIG. 1 shows a block diagram of the various components of an embodiment of a thermography system. As shown, the system 10 comprises a thermography catheter 12 capable of being inserted into a blood vessel of a patient 14. The catheter 12 is connected to a data interface front end 18 through an interface cable 16. The embodiment shown is capable of attaching to a variety of thermography catheters 12, including, for example, thermographic balloon catheters as disclosed in commonly owned U.S. Pat. No. 6,245,026, filed Jul. 1, 1999, titled “Thermography Catheter”, thermographic basket catheters as disclosed in U.S. patent application Ser. No. 09/938,963, filed Aug. 24, 2001, titled “Thermography Catheter with Flexible Circuit Temperature Sensors”, and other thermographic catheters as disclosed in U.S. Pat. No. 5,871,449, filed Dec. 27, 1996, titled “Device and Method for Locating Inflamed Plaque in an Artery”, U.S. patent application Ser. No. 09/246,603, filed Feb. 8, 1999, titled “System for Locating Inflamed Plaque in an Artery”, and U.S. Pat. No. 5,924,997, filed Jul. 17, 1997, titled “Catheter and Method for the Thermal Mapping of Hot Spots in Vascular Lesions of the Human Body”, each of which is hereby incorporated by reference in its entirety herein.

As shown in FIG. 1, themography catheter 12 may be capable of measuring blood temperature within a blood vessel, arterial wall temperature, or both. The data interface front end 18 is connected to and communicates with the system controller or CPU 20 attached to a power supply 22. The system controller 20 monitors and controls the operation of the system 10. The system controller 20 may include a personal computer or other computer devices. A keyboard 24 and/or a pendant function device 26 may be attached to the system controller 20 thereby permitting the user to input information into the system controller 20. The system controller 20 is attached to a display 28 and may also be in communication with at least one storage device 30. Exemplary storage devices 30 may include volatile and non-volatile memory devices or CD-ROMs. In an alternate embodiment, the storage device 30 may comprise an external computer of storage unit accessible through a communication port located within the storage device 30. An encoder 32 having a pullback device 34 may be attached thereto is connected to the system controller 20. The system controller 20 is capable of controllably moving or articulating the catheter 12 in an axial direction within a blood vessel. In the illustrated embodiment, an expansion module 36 may be included. The system controller 20 may also have other output ports 38′ to enable the system to be coupled to alternative video displays, imaging sytstems, such as MRI, angiography systems and the like, and computer or internet networks in order to send and receive data from other sites.

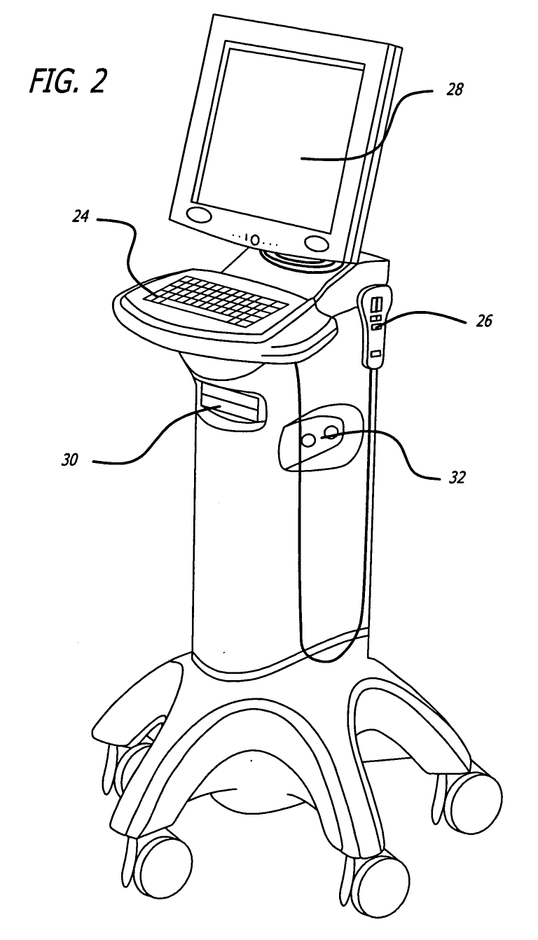

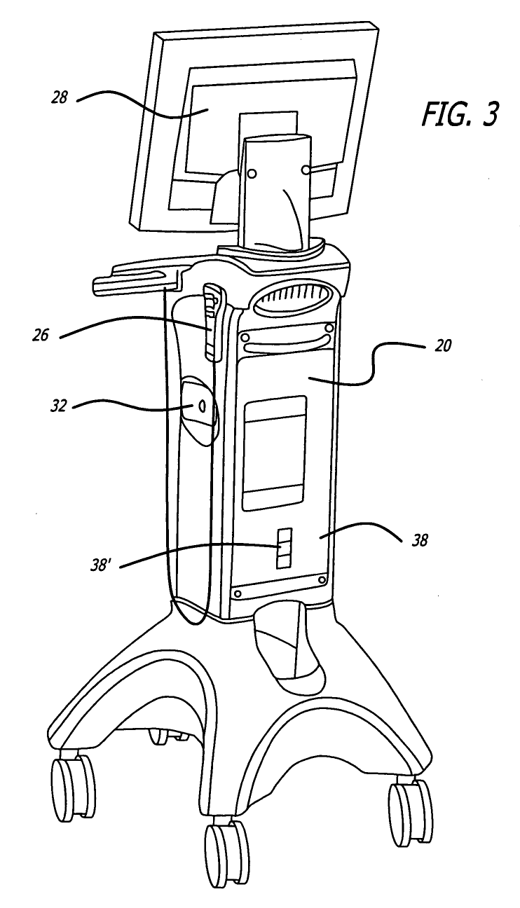

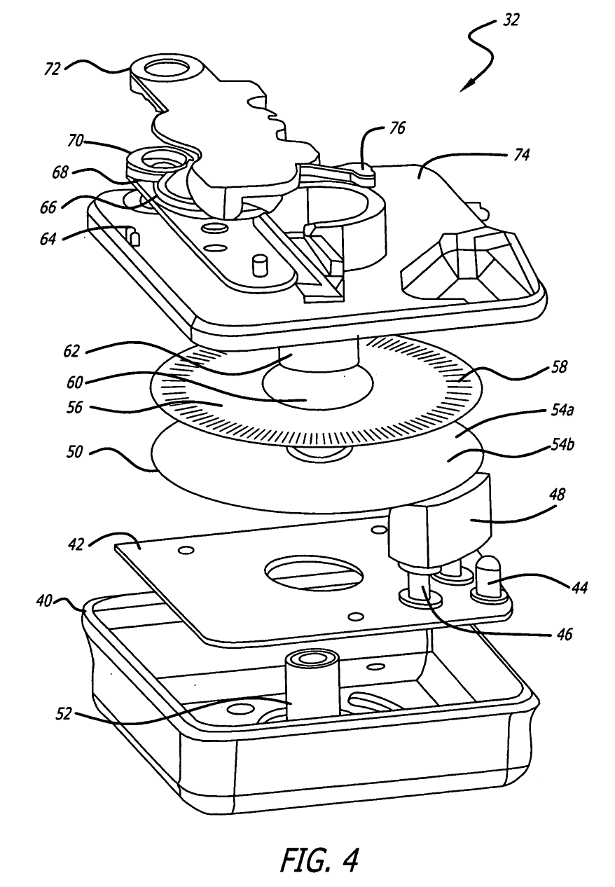

The expansion module 36 permits a variety of other devices to be connected to the system 10. For example, an intravascular ultrasound (IVUS) device and/or an agent delivery device may be coupled to the thereby permitting the localized delivery of therapeutics agents to an area of interest. FIGS. 2 and 3 show exterior views of one embodiment of a thermography instrument that has a pedestal supported by wheels which allow the instrument to be readily moved about a medical suite. As shown in FIG. 2, the keyboard 24 and storage device 30 are positioned proximate to the display 28. The pendant function key 26 may be added if desired. As shown in FIG. 3, the encoder 32 is attached to the system controller 20. In addition, the system controller 20 may include a potential equalization terminal 38 to enable calibration of the system controller 20. The illustrated embodiment shown in FIGS. 2 and 3 is not intended to limit the present invention in function or appearance.

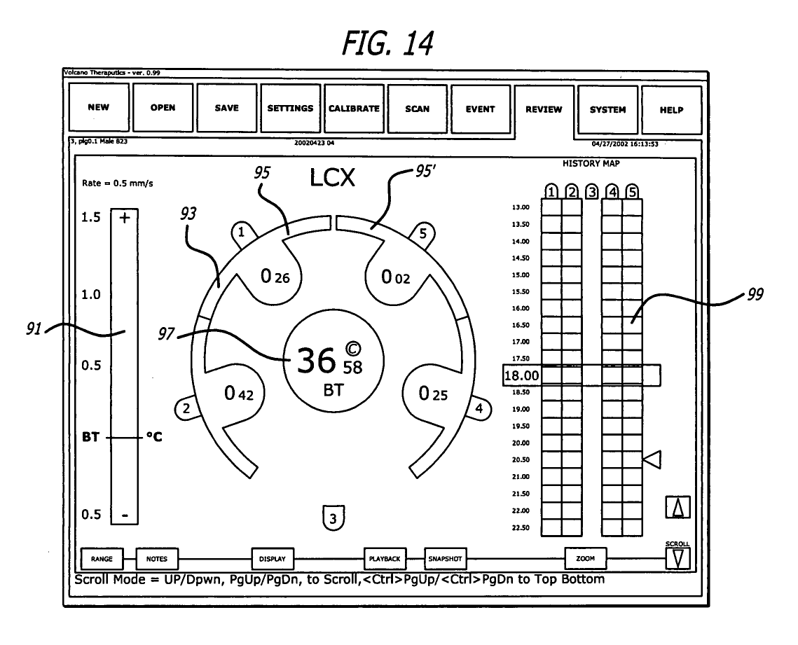

FIG. 14 shows a detailed view of the encoder 32. The encoder 32 comprises a case bottom 40 capable of housing the various component of the encoder 32. An encoder circuit board 42 may be positioned within the case bottom 40. The circuit board 42 may include at least one microprocessor (not shown), at least one emitter, such as a energy emitter, specifically, a photo or infrared (IR) emitter 44, at least one energy detector, such as a photo detector, specifically, an IR detector 46, and may include at least one connector (not shown) for connecting the encoder circuit board 42 to a signal source and/or power supply. In the illustrated embodiment the encoder circuit board 42 includes one IR emitter 44 and two IR detectors 46 optically isolated from the IR emitter by a detector shield 48. An encoder mask 50 may be positioned on a center post 52 attached to the case bottom 40. The encoder mask 50 includes at least two slits 54a, 54b formed therein. An encoder disk 56 having a plurality of slits 58 formed therein is positioned on the center post 52 adjacent to the encoder mask 50. The slits 58 formed on the encoder disk 56 are 90 degree out of phase with the slits 54a, 54b formed on the encoder mask 50. A drive wheel 60 may be coupled to the encoder disk 56 with a drive sleeve 62.

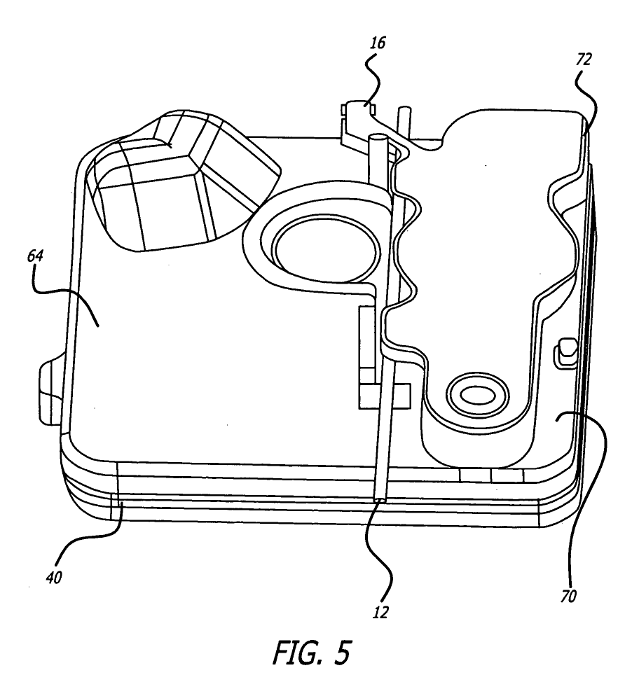

A case top 64 encloses the various components of the encoder 32 within the a protective housing formed by the case top 64 and case bottom 40. In addition, the interior surface of the case top 64 may include a reflective material capable of reflecting light from the at least one IR emitter 44 through the slits 54a, 54b, and 58 formed on the encoder mask 50 and encoder disk 56. A tension wheel 66 and tension O-ring 68 are positioned between bottom tension arm 70 and top tension arm 72. The tension arms 70 and 72 are capable of rotating about the center post receiver 74 formed in the case top 64, thereby permitting the catheter 12 to be positioned therebetween. The amount of tension applied to the catheter 12 by the tension arms 70 and 72 may be adjusted by actuating a latch 76 positioned on the tension arms 70 and 72. FIG. 5 shows the encoder 32 of the present invention engaging a portion of the catheter 12.

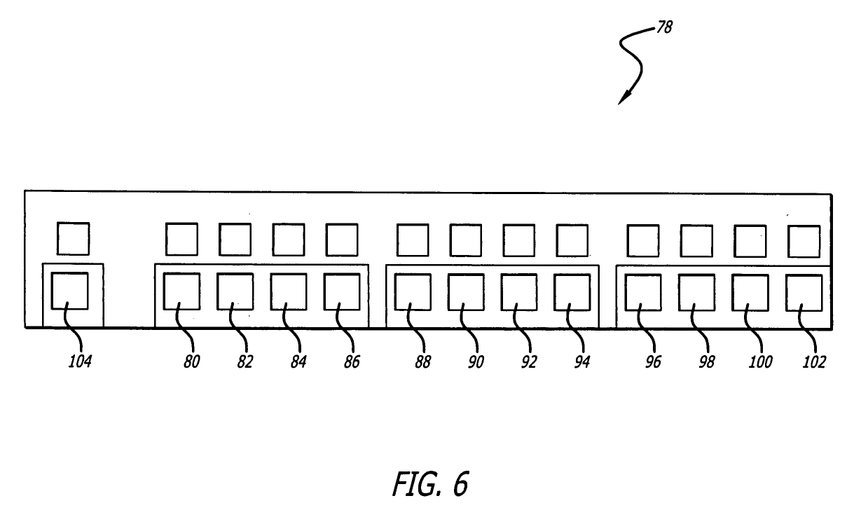

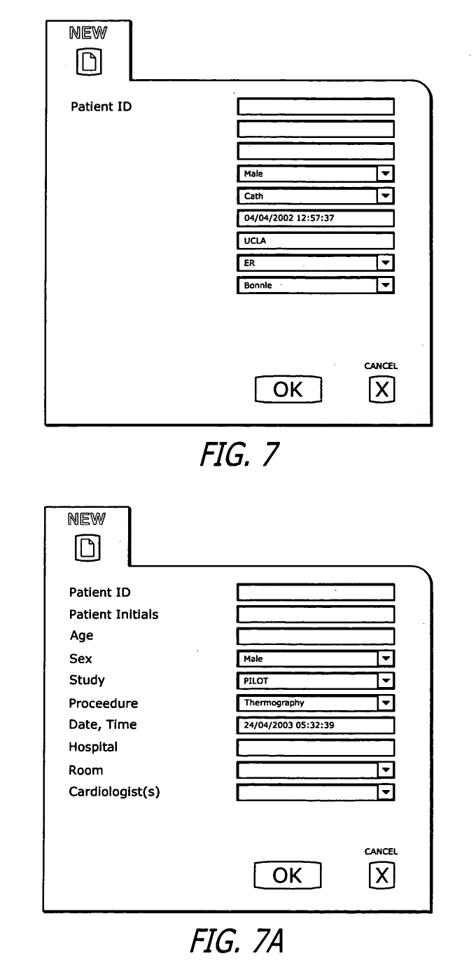

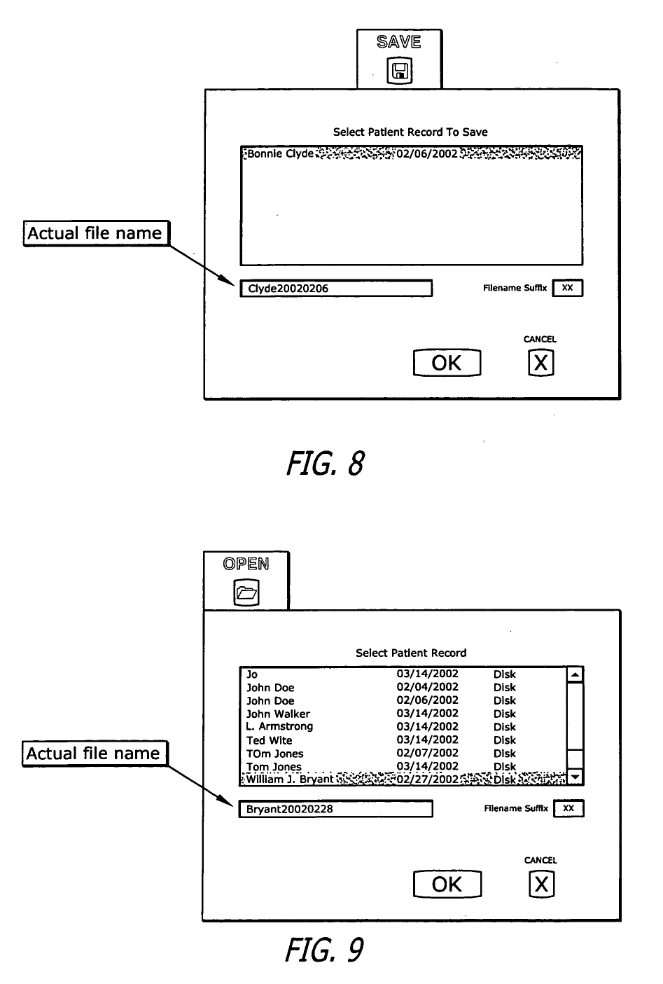

A method of identifying and/or treating vulnerable plaque in vivo using the device described above is also disclosed. Prior to initiating a thermal mapping procedure, the user may input patient specific information into the system 10 with the keyboard 24. FIG. 6 shows the function actuators 78 located on the keyboard 24. As shown, the function actuators 78 may include NEW 80, OPEN 82, SAVE 84, SETTINGS 86, CALIBRATE 88, SCAN 90, EVENT 92, REVIEW 94, SYSTEM 96, HELP 98, BACK 100, OK 102, and CANCEL 104 actuators. The system 10 may display a graphical user interface (GUI) on the display 28. FIGS. 7 and 7A show exemplary GUI display screens that may be shown on the display 28. The user may enter or be prompted to enter patient specific information into the system controller 20. For example, FIG. 7 shows a GUI display which may be shown when actuating the NEW actuator 80. Thereafter, the user may enter information into to the appropriate fields. If desired the user may save then enter information to the storage device 30 by actuating the SAVE actuator 84. FIG. 8 shows an exemplary GUI display of the save screen. In an alternate embodiment, the user may access preexisting patient history files stored within the storage device 30 or in an external memory device accessible through a communication port. To access previous saved information the user may actuate the OPEN actuator 82 thereby displaying the OPEN GUI screen as shown in FIG. 9.

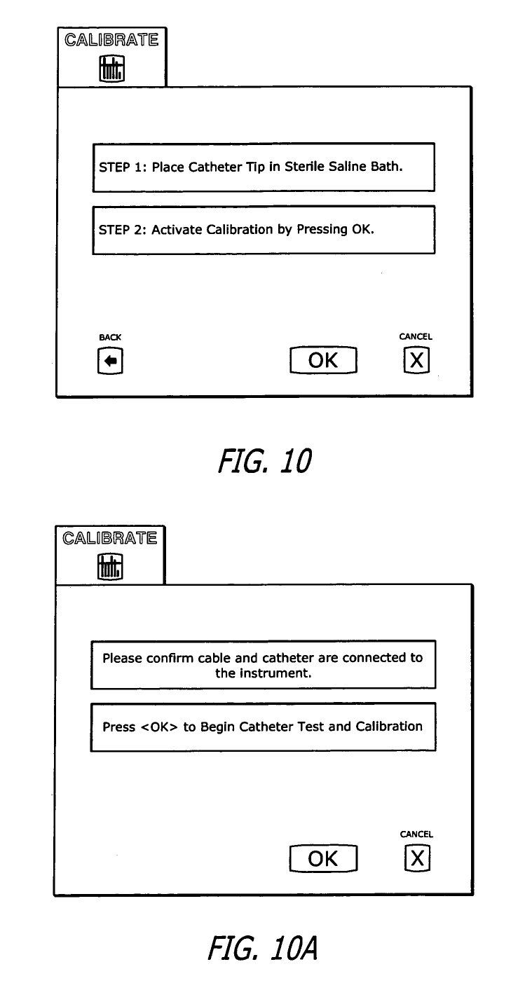

Prior to commencing a procedure, the system 10 may be calibrated. The calibration process may include a two step procedure wherein the system controller 20 and the catheter 12 may be individually calibrated. To calibrate the system controller 20 the user may actuate the CALIBRATION actuator 88 on the keyboard 24 (see FIG. 6). Thereafter, the user installs a grounding plug (not shown) into a potential equalization terminal 38 (shown in FIG. 3), thereby grounding the various components of the system controller 20. In an alternate embodiment, the system 10 may include an internal grounding device (not shown) capable of internally grounding the various components of the system controller 20. To calibrate the catheter 12, the user may connect the catheter 12 to the system controller 20 and obtain a thermal reading within a fluid having a known temperature. For example, the user may insert the catheter 12 into a saline solution of a known temperature. Thereafter, the user may compare the measured value with the known value. If desired, the user may save the results of the calibration procedure within the storage device 30. FIGS. 10 and 10A show graphical user interface (GUI) screens of a calibration procedure as displayed on the display 28. Once completed the user may actuate the OK actuator 102 on the function actuator 78. Alternative calibration procedures are described in U.S. Provisional patent application Ser. No. 60/431,326, filed Dec. 6, 2002, which is incorporated by reference herein in its entirety.

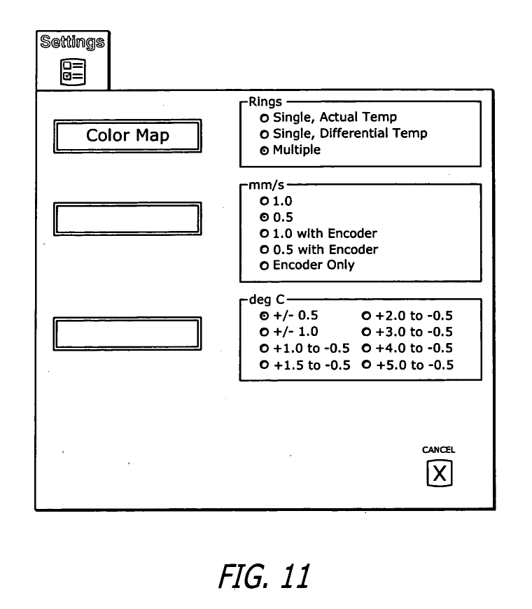

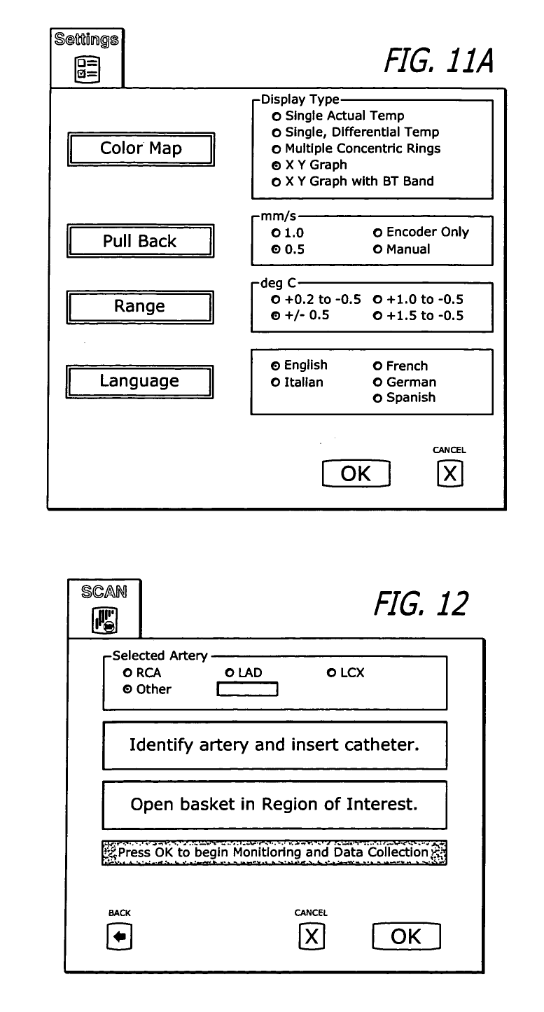

With the system 10 calibrated and the patient information entered into the system, the user may set the scanning settings of the present invention by actuating the SETTINGS actuator 86 (see FIG. 6) on the keyboard 24. FIGS. 11 and 11A show GUI screens of the settings adjustment process. As shown in FIGS. 11 and 11A, embodiments of the present invention permit the user to tailor the scanning process as desired. For example, the user may tailor the thermal measurement range. In an alternate embodiment, the user may adjust the pull back speed of the catheter.

The catheter 12 may be inserted into a blood vessel of a patient using standard percutaneous procedures. Thereafter, the catheter 12 may be inserted therein and advanced through the circulatory system to a location past an area of interest. If desired, the catheter may include IVUS or other imaging devices thereon thereby permitting the user to precisely position the catheter 12 within the blood vessel. Once positioned, the user may actuate the thermal catheter thereby permitting the thermal measuring device located thereon to contact or become positioned proximate to the vessel wall. The user may then actuate the SCAN actuator 90 (see FIG. 6) on the keyboard 24. FIG. 12 shows the GUI display of the scan screen. During the measurement process the pull back device 34 attached to the encoder 32 retracts the catheter 12 through the blood vessel and a pre-determined rate. Alternatively, the catheter 12 may be distally advanced past an area of interest within a body vessel while temperature or other thermography data is being measured. During the retraction process the thermal sensors located on the catheter 12 measure the temperature of the vessel wall or blood fluid at a pre-determined rate and frequency. If desired, the user may return the catheter 12 to the starting location and re-initiate the procedure. Prior to removing the catheter 12 from the blood vessel, the user may deliver a therapeutic agent to an area of interest with the catheter 12.

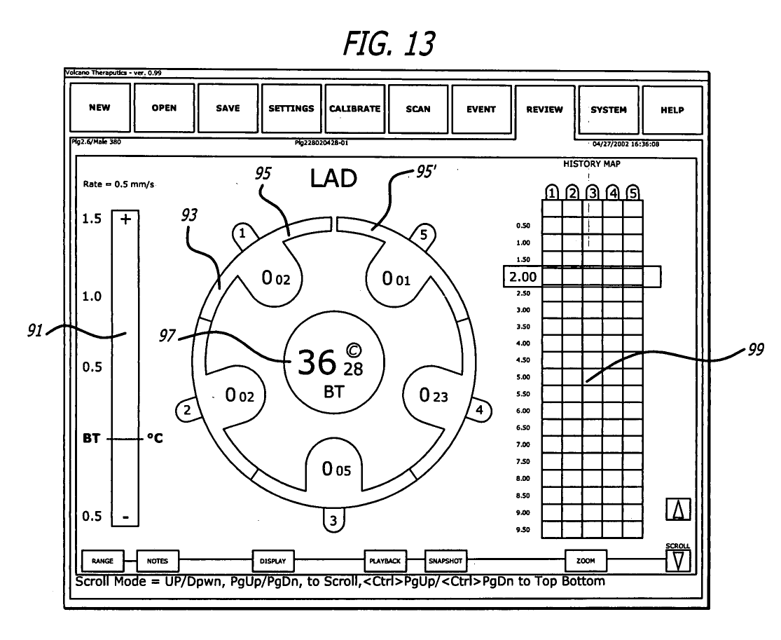

During the measurement process, the system 10 may display the measured results on the display 28. The measured results may be illustrated in a plurality of ways, including, for example, bar graph, two-dimensional chart, and a three-dimensional image. FIGS. 13-16 illustrate graphical displays of a measurement procedure as illustrated on the display 28. FIGS. 13-16 show a graphical representation of measuring process using a thermography catheter having five thermal sensor suites 1, 2, 3, 4, and 5 circumferentially positioned thereon to measure the temperature within a vessel. A thermal scale 91 is displayed proximate to the sensor map 93. The sensor map 93 is displayed as a circumferential annular ring 95 which is broken into distinct circumferential sections 95′ which display thermography data from corresponding thermal sensors. All of the graphical displays of thermography data discussed herein may be displayed as numerical data corresponding to temperatures or color data in which the color displayed is a function of the temperature detected or calculated. The temperature data displayed for any of these embodiments may be a measurement of an absolute temperature, or it may be a difference in temperature such as a measurement of the difference in the temperature of fluid in a body vessel and the temperature of the body vessel wall adjacent the blood or some other desired parameter or measurement. As such, the thermal scale 91 may include a reference temperature, such as a blood temperature BT 97. Similarly, a history map or matrix 99 is also displayed wherein thermal readings received from each thermal sensor at various time periods and/or locations may be recorded.

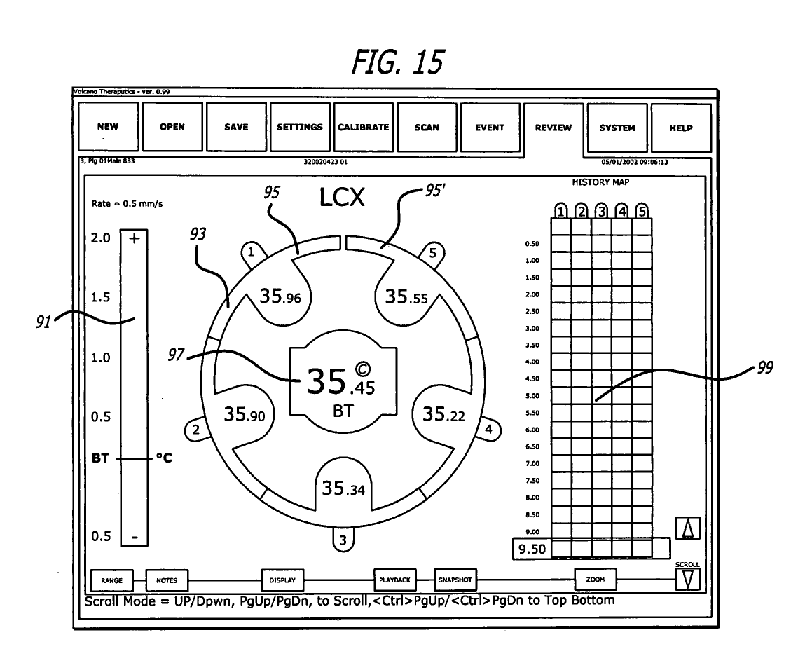

FIG. 13 shows a measurement display wherein the sensors 1-5 have recorded thermal readings at or below the blood temperature BT 37. FIG. 14 shows a measurement display wherein sensors 1, 3, 4, and 5 have recorded thermal readings at or below the blood temperature BT, and sensor 2 has recorded thermal readings above the blood temperature BT. FIG. 15 shows a measurement display wherein sensors 3-5 have recorded thermal readings at or below the blood temperature BT, and sensors 1-2 have recorded thermal readings above the blood temperature BT. The measured values may be saved to the storage device 30 if desired. Furthermore, the user may actuate the EVENT actuator 92 on the function actuator (see FIG. 6) to highlight the thermal readings at a specific area or time. FIGS. 13-15 show various highlighted event regions.

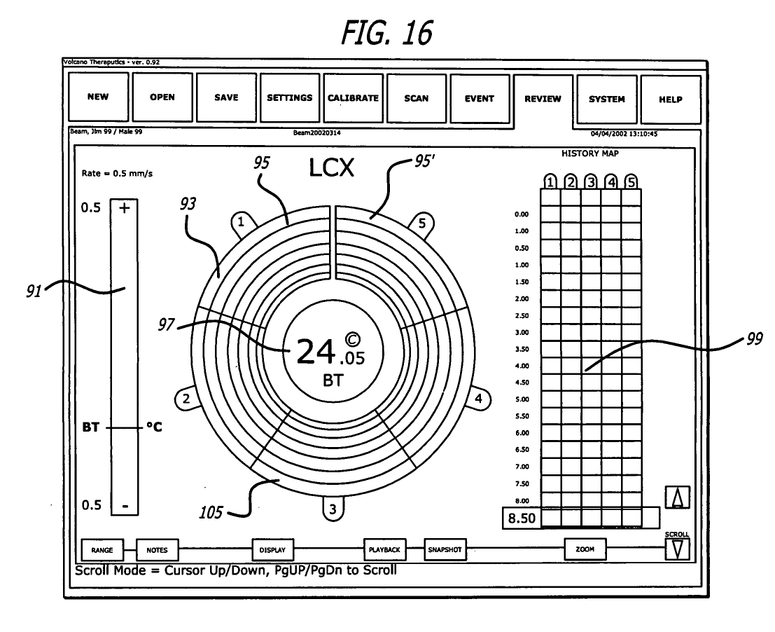

FIG. 16 illustrates a display having a sensor map 93 in which thermography data is displayed as a plurality of concentric annular rings 95, each of which is broken into distinct circumferential sections 95′ which display data from a corresponding thermography data input or data source, such as thermal sensors of a thermography catheter. Each concentric ring 95 can represent a different data point during a thermography procedure. For example, for a procedure in which a thermography catheter, such as the catheter 110 described below, is being withdrawn or axially translated in a proximal direction, an outer-most ring 105 may be used to display most recently sampled thermography data from thermal sensors of the thermography catheter and the inner-most ring 106 can be used to display the earliest taken thermography data. This method can be used to generate a tunnel-like view of data which results in a visual thermal map that can be readily interpreted by an operator of the thermography system. A similar process may be used if the catheter 110 is axially translated in a forward or distal direction.

FIG. 16A shows another embodiment of display with a sensor map 175 wherein thermography data from five thermal sensors is displayed as a graph 176 having location or distance on a first axis 177 and temperature and temperature differential on a second axis 178. Each plot 181 corresponding to a thermal sensor can be color coded with a color indicated in legend column 182 to the right of the graph 176, which may also show blood temperature shown at the top of the column 182. The graph display 176 readily indicates significant changes in thermography data for a particular axial location or zone, such as the peak 183 indicated at position 13 along the first axis 177. The rate of axial displacement is indicated on the display, as well as patient data of interest to procedure. Other user options for the display of FIG. 16A can be the same as those described above with regard to other display embodiments.

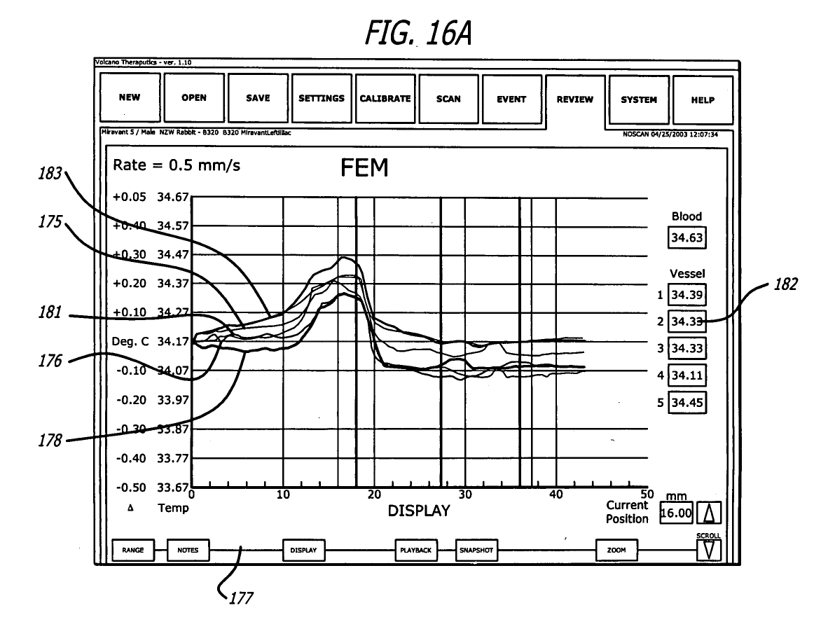

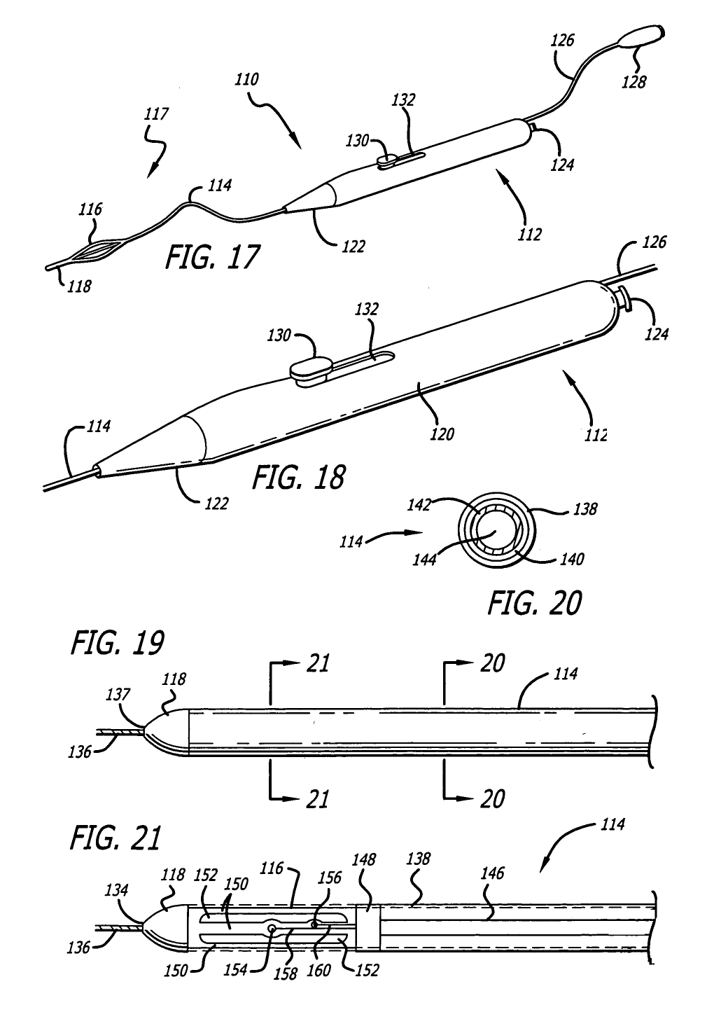

FIG. 17 shows an embodiment of a thermography catheter 110. As shown, the thermography catheter 110 is comprised of a handle 112 coupled to or otherwise in communication with an elongated body 114. An expandable slotted body 116 may be positioned on a distal section 117 proximate to the distal end 118 of the elongated body 114. As shown in FIGS. 17 and 18, the handle 112 may include a handle body 120 having an elongated body receiver 122 attached thereto and a guidewire port 124 formed thereon. The elongated body receiver 122 is capable of receiving the elongated body 114 therein. In the illustrated embodiment the elongated body receiver 122 is detachably coupled to the handle body 120. In an alternate embodiment the elongated body receiver 122 may be integral to the handle body 120. The guidewire port 124 may be capable of receiving at least one guidewire therein and may be in communication with the central shaft 142 formed in the elongated body 114 (see FIG. 20). A sensor coupler 128 may be coupled to the at least one sensor conduit 126 which may permit the thermography catheter to be connected to or otherwise communicate with various analyzing devices (not shown), including, for example, computers, display devices, amp meters, ohm meters, electromagnetic analyzers, and blood analyzers. An elongated body actuator 130 may be slidably positioned within an actuator recess 132 formed on the handle body 122. The thermography catheter 110 may be manufactured from a variety of materials in a variety of lengths and diameters.

FIGS. 19-21 show various illustrations of the elongated body 114 in a non-deployed state. FIG. 19 shows the elongated body 114 prior to actuation wherein the elongated body 114 is engaging the distal tip 118. The distal tip 118 may include a guidewire port 134 capable of receiving a guidewire 136 therein. As shown in FIG. 20, the elongated body 114 may include a movable outer sleeve 138 forming a sleeve lumen 140 housing an central shaft 142 therein. In a non-deployed state, the expandable slotted body 116 of the thermography catheter 110 may be positioned within the sleeve lumen 140 formed by the movable outer sleeve 138. FIG. 21 illustrates the position of the expandable slotted body 116 within the sleeve lumen 140 prior to deployment. As shown, the expandable slotted body 116 may be compressed inwardly by the movable outer sleeve 138 and may be positioned within the sleeve lumen 140. The central shaft 142 defines at least one internal passage 144 therein. In the illustrated embodiment a single internal passage 144 is formed in the central shaft 142, however, central shaft 142 may define a plurality of internal passages therein. The internal passage 144 formed within the central shaft 142 may be capable of receiving the guidewire 136 (see FIG. 19).

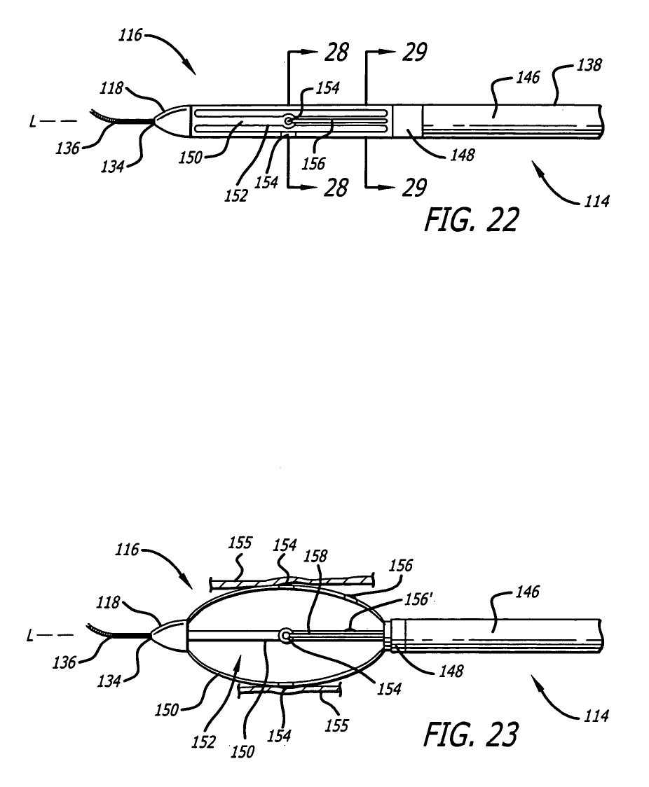

FIGS. 21-23 show various illustrations of the expandable slotted body 116 during various stages of use. FIGS. 21 and 22 show the expandable slotted body 116 located within the sleeve lumen 140 in a non-expanded state prior to deployment. As shown, a deployment support member 148 may be positioned within or proximate to the internal passage 144 formed within the central shaft 142 (see FIG. 20). In one embodiment, the deployment support member 148 includes an aperture (not shown) sized to receive the guidewire lumen 146 therethrough. In the illustrated embodiments the deployment support member 148 is positioned proximate to the expandable slotted body 116. In an alternate embodiment the deployment support member 148 may be positioned at various locations on or within the elongated body 114 or the handle 112 (see FIG. 17).

The expandable slotted body 116 may be comprised of one or more slotted body arms 150 separated by one or more slots 152. The expandable slotted body 116 may be generally hollow in design, thereby defining an inner lumen (not shown) capable of receiving the guidewire 136 or the guidewire lumen 146 therethrough. In an alternate embodiment, the expandable slotted body 116 may comprise a hypodermic tube having one or more slots 152 formed therein, thereby defining one or more slotted body arms 150 thereon. The expandable slotted body 116 may be manufactured from a variety of materials, including, for example, Nitinol and other shape memory alloys (SMA), steel including stainless steel and other alloys, titanium, polymers, composite materials, and like materials. In the illustrated embodiment, the one or more slotted body arms 150 are attached to the deployment support member 148. The one or more slotted body arms 150 may be adhesive coupled to the deployment support member 150 using, for example, 205-CTH epoxy or any other biologically compatible adhesive. During manufacture, the one or more slotted body arms 150 are formed in a deployed position in relaxed state as shown in FIG. 7, wherein the one or more slotted body arms 50 are flared outwardly from the longitudinal axis L of the expandable slotted body 116.

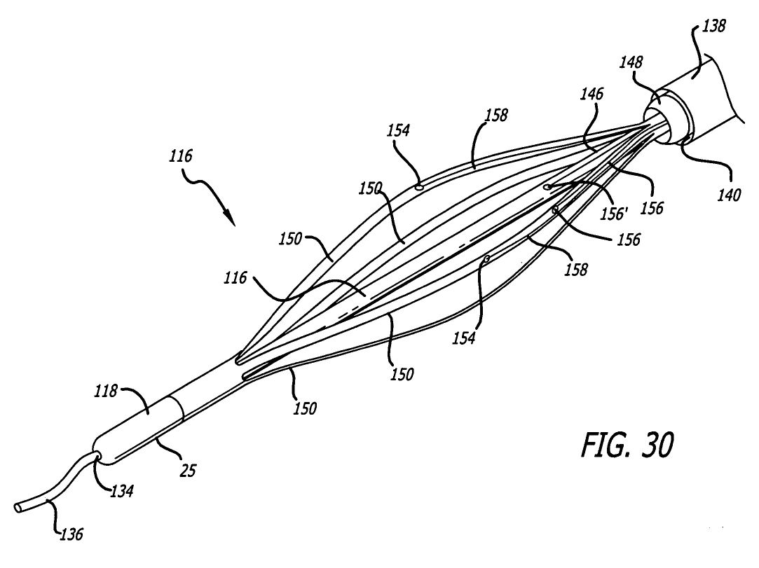

One or more sensors may be positioned on the one or more slotted body arms 150. Exemplary sensors include, without limitation, ultrasonic sensors, flow sensors, thermal sensors, blood temperature sensors, electrical contact sensors, conductivity sensors, electromagnetic detectors, chemical sensors, and infrared sensors. As such, the thermography catheter 110 may be capable of simultaneously examining a number of characteristics of tissue within the body of a patient, including, for example, vessel wall temperature, blood temperature, fluorescence, luminescence, flow rate, and flow pressure. As shown in FIGS. 21-23, the one or more support members 150 of the expandable slotted body 116 may include one or more vessel wall temperature sensors 154 and one or more blood temperature sensors 156 thereon, thereby permitting the user to measure vessel wall temperature and blood temperature simultaneously. As shown in FIG. 23, the one or more vessel wall temperature sensors 154 may be positioned on or near the apex of the arcuate slotted body arms 150 when the expandable slotted body 116 is deployed in an expanded state, thereby permitting the one or more vessel wall temperature sensors 154 to contact the vessel wall 155. FIG. 23 also shows a temperature sensor 156′ located on the guidewire lumen 146 which may be used in conjunction with blood temperature sensor 156 or as an alternative to blood temperature sensor 156 disposed on the support member or slotted body arm 150. Blood temperature sensor 156′ is also shown in FIG. 30 in perspective.

Similarly, the one or more blood temperature sensors 156 may be positioned on the one or more slotted body arms 150 at any radial distance less than the radial distance of the apex of the arcuate slotted body arms 150 relative the longitudinal axis L of the expandable slotted body 16 when the expandable slotted body 116 is in a deployed state, thereby preventing the one or more blood temperature sensors 154 from contacting the vessel wall 155 when the expandable slotted body 116 is deployed to an expanded state. As a result, the one or more blood temperature sensors 154 may be thermally isolated from the one or more vessel wall temperature sensors 154 thereby enabling the real time measurement of vessel wall temperature and blood temperature. For example, the one or more blood temperature sensors 156 may be located proximate to the deployable support member 148 or the distal tip 118 to ensure the one or more blood temperature sensors 156 do not contact the vessel wall 155 during blood temperature measurement. As shown in FIG. 21, at least one vessel wall sensor conduit 158 is located on or proximate to the one or more slotted body arms 150 and is attached to or otherwise in communication with the one or more vessel wall temperature sensors 154. Similarly, at least one blood temperature conduit 160 is located on or proximate to the one or more slotted body arms 150 and is attached to or otherwise in communication with the one or more blood temperature sensors 156.

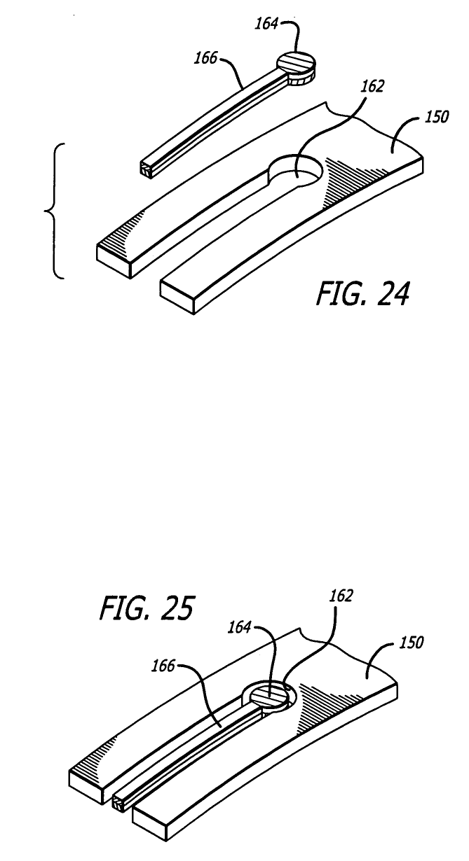

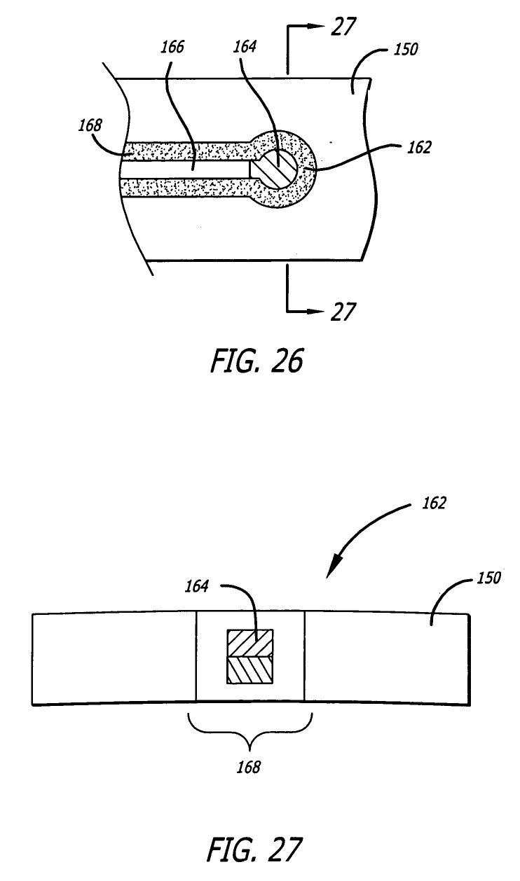

FIGS. 24-27 show various detailed illustrations of a slotted body arm 150 of the thermography catheter having a sensor slot 162 formed therein. As shown in FIGS. 24-27, the sensor slot 162 may be longitudinally positioned along the slotted body arm 150 and may be capable of receiving a thermocouple or other sensor device 164 therein. The thermocouple or other sensor device 164 may communicate via one or more conduits 166 attached to or integral with at least one of the vessel wall sensor conduit 158 or the blood temperature conduit 160, and may be in communication with at least one external detection device (not shown) attached to the sensor coupler 128 (see FIG. 21). As shown in FIGS. 26 and 27, the thermocouple or other sensor device 164 may be adhesively attached to the slotted body arm 150 within the sensor slot 162 with an epoxy or other biological compatible adhesive material 168, thereby reducing the profile of the expandable slotted body 116 when compared to prior art devices. An example of such a device is disclosed in patent application Ser. No. 10/099,409, filed Mar. 15, 2002, which is incorporated by reference in its entirety herein. As a result, the thermography catheter may be effectively used in smaller diameter locations within the body as compared with prior art systems. The sensor slot 162 may be formed in the slotted body arm 150 by laser etching or chemically etching the outer surface of an expandable tube or sheet, prior to forming each of the individual slotted body arms 150 that make up the final expandable slotted body 150.

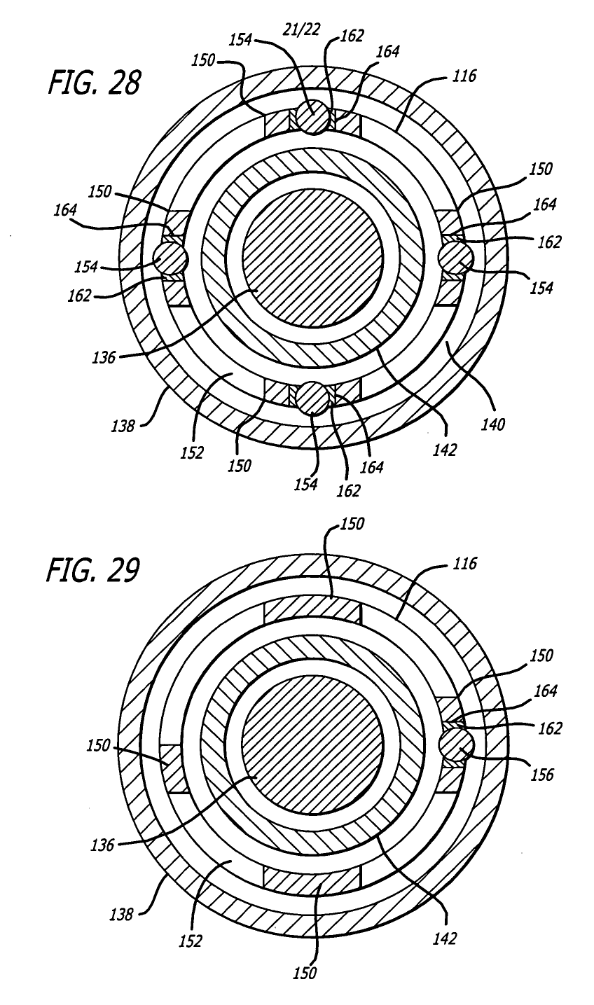

FIGS. 28-29 show various cross-sectional views of the expandable slotted body 116 in a non-deployed state. FIG. 28 shows a cross-sectional view of the midsection of the expandable slotted body 116 positioned within the movable outer sleeve 138 in a non-deployed state. As shown, at least one vessel wall temperature sensor 154 is positioned within each sensor slot 162 formed in the slotted body arms 150 and may be coupled to the slotted body arms 150 using epoxy 164. The vessel wall temperature sensors 154 may be positioned on the slotted body arms 150 to enable the vessel wall temperature sensors 154 to contact the internal vessel wall during the measurement process, thereby resulting in more accurate thermal measurements of wall tissue positioned proximate thereto. The guidewire lumen 142, containing the guidewire 136 therein, is positioned within and traverses through the expandable slotted body 116. FIG. 29 shows a cross-sectional view of the expandable slotted body 116 positioned within the movable outer sleeve 138 in a non-deployed state. As shown, at least one blood temperature sensor 156 is positioned within a sensor slot 162 formed in at least one of the slotted body arm 150 may be and coupled to the slotted body arm 150 using epoxy 164. The blood temperature sensor 156 may be positioned on the slotted body arm 150 incident to a blood flow through the vessel and thermally isolated from the vessel wall, thereby permitting the real time, simultaneous measurement of blood temperature and vessel wall temperature.

FIG. 30 shows a perspective view of the expandable slotted body 116 of the present invention during use. As shown, the one or more slotted body arms 150 expand radially outwardly from the longitudinal axis L of the expandable slotted body 116, thereby permitting the one or more vessel wall temperature sensors 154 to contact the internal surface of the vessel wall 155 to be examined. Similarly, the one or more blood temperature sensors 156 are positioned on the one or more slotted body arms 150 such that the one or more blood temperature sensors 156 are prevented from contacting the vessel wall 155, thereby thermally isolating the one or more blood temperature sensors 156. Blood temperature sensor 156′ is also shown on the guidewire lumen 146 in a position that would isolate the blood temperature sensor 156′ from contact with the vessel wall 155. As shown, a guidewire lumen 146 exits through the deployment support member 148 positioned within the movable outer sleeve 38 and traverses along the longitudinal axis L of the expandable slotted body 116, eventually connecting to the guidewire port 134 formed in the distal tip 118. In the illustrated embodiment four slotted body arms 150 are expanded outwardly thereby forming a “basket” catheter, although the thermography catheter may include any number of slotted body arms 150.

In another embodiment, at least one of the vessel wall temperature sensors 154 or the blood temperature sensors 156 may be comprised of flexible circuits integrated into slotted body arms 150. A particular flexible circuit that is applicable to the thermography catheter is disclosed in commonly assigned U.S. patent application Ser. No. 09/938,963, which is incorporated herein by reference.

In one embodiment, the flexible circuit is comprised of polymer thick film flex circuit that incorporates a specially formulated conductive or resistive ink that is screen printed onto the flexible substrate to create the thermal sensor circuit patterns. This substrate is then adhered to the surface of each of the slotted body arms 150. In an alternate embodiment, the substrate can be adhered to independently expandable, resilient body arms which are not part of an expandable slotted body. As with all of the embodiments, the thermography catheter 110 may be provided with any number of slotted body arms, such as four, five, six, or more.

During use, a guidewire 136 (see FIG. 19) is introduced into the blood vessel of a patient. Typically, access to the blood vessel may be obtained by forming an incision within the patient's skin proximate to a blood vessel. Similarly, an incision may be made in the blood vessel. Once the guidewire 136 is positioned with the blood vessel, the thermography catheter 110 is attached to the guidewire 136 and the distal tip 118 of the thermography catheter 110 (see FIG. 19) is introduced into the blood vessel of a patient and advanced over the guidewire 136 to the area of interest. The thermography catheter 110 may include IVUS or other imaging devices thereon thereby permitting the user to precisely position the thermography catheter 110 within the blood vessel. In one embodiment, the distal tip 118 of the thermography catheter may be advanced through the blood vessel to a position distal of the area of interest. The expandable slotted body 116 may be positioned within the movable outer sleeve 138 (see FIG. 21) when introduced into the blood vessel. Thereafter, the user operates the actuator 130 located on the handle 112 to a deployed positioned within the actuator recess 132 (see FIG. 17). The rearward operation of the actuator 130 positioned on the handle 112 (see FIG. 17) results in the movable sleeve 138 retracting rearwardly, thereby exposing the expandable slotted body 116 and permitting the expandable slotted body 116 to move to return to a relaxed, expanded state wherein the one or more slotted body arms 150 flare outwardly (see FIG. 23).

As a result, the at least one vessel wall temperature sensor 154 located on the one or more slotted body arms 150 contacts the vessel wall 155 thereby enabling the measurement of the vessel wall temperature. Simultaneously, the at least one blood temperature sensor 156 located on the one or more slotted body arms 150 measures the blood temperature without contacting the vessel wall 155 (see FIG. 23), thereby permitting the real time measurement of vessel wall temperature and blood temperature. Thereafter, the distal section 117 of the thermography catheter 110 is retracted proximally through the blood vessel while simultaneously measuring vessel wall temperature and blood temperature. The vessel wall temperature and blood temperature measurements are sent to a analyzer (not shown) via the vessel temperature conduit 158 and the blood temperature conduit 160. Thereafter, the user returns the actuator 130 located on the handle 130 to a non-deployed position within the actuator recess 132. As a result, the movable outer sleeve 138 advances towards the distal tip 118 (see FIG. 19). While advancing towards the distal tip 118, the movable outer sleeve engages the expandable slotted body 116, which is compresses into the sleeve lumen 140, thereby returning the expandable slotted body 116 to a non-deployed state (see FIG. 19). Prior to removing the thermography catheter 110 from the blood vessel, the user may delivery a therapeutic agent to an area of interest with the thermography catheter 110. Thereafter, the thermography catheter 110 and the guidewire 136 may be removed from the patient and the entry incisions may be closed.

While illustrative embodiments have been described above, it is understood that various modifications will be apparent to those of ordinary skill in the art. Many such modifications are contemplated as being within the spirit and scope of the invention.

What is claimed is:

1. A thermography system, comprising:
a thermography catheter having a thermal sensor on a distal section thereof;
a system controller coupled to the thermal sensor; and
a display configured to graphically display thermography data from the thermal sensor.

2. The thermography system of claim 1 wherein the display comprises a graphic user interface.

3. The thermography system of claim 1 wherein the thermography catheter comprises a plurality of thermal sensors in a substantially annular array and the display is configured to display thermography data from the sensors in a series of concentric rings each of which are divided into circumferential sections with each circumferential section correlating to a distinct thermal sensor and with each concentric ring representing a different thermography data point.

4. The thermography system of claim 3 wherein display comprises about 3 to about 20 circumferential sections.

5. The thermography system of claim 3 wherein the display comprises about 4 to about 8 circumferential sections.

6. The thermography system of claim 3 wherein each circumferential section displays a color that is a function of the temperature of the thermal sensor corresponding to the circumferential section.

7. The thermography system of claim 3 wherein difference between blood temperature and the temperature of a wall of a body vessel adjacent the blood is displayed in a circumferential section.

8. The thermography system of claim 3 wherein blood temperature is displayed in a center portion of the display.

9. The thermography system of claim 1 wherein the thermography catheter comprises a plurality of thermal sensors in a substantially annular array and wherein thermography data from the thermal sensors is displayed in an annular ring on a screen of the display that is divided into circumferential sections with each section corresponding to a thermal sensor.

10. The thermography system of claim 9 wherein each circumferential section displays a color that is a function of the temperature of the thermal sensor corresponding to the circumferential section.

11. The thermography system of claim 9 wherein difference between blood temperature and the temperature of a wall of a body vessel adjacent the blood is displayed in a circumferential section.

12. The thermography system of claim 9 wherein blood temperature is displayed in a center portion of the annular ring.

13. The thermography system of claim 1 wherein the system controller comprises a CPU.

14. The thermography system of claim 1 wherein the thermal sensor is coupled by a conductor to a connector at a proximal end of the catheter and the connector is coupled to the system controller by an interface cable.

15. The thermography system of claim 1 further comprising a pull back device configured to couple to a proximal portion of the catheter and exert an axial force on the catheter.

16. The thermography system of claim 15 further comprising a position encoder coupled to the pull back device and coupled to the system controller wherein the position encoder is configured to transmit position data of the catheter to the system controller.

17. A thermography system, comprising:
a thermography catheter having a thermal sensor on a distal section thereof and a plurality of thermal sensors in a substantially annular array;
a system controller coupled to the thermal sensors; and
a display configured to graphically display thermography data from the thermal sensor in a series of concentric rings divided into circumferential sections with each circumferential section correlating to a distinct thermal sensor and with adjacent rings representing different thermography data points.

18. The thermography system of claim 17 wherein the display comprises a graphic user interface.

19. A thermography system, comprising:
a thermography catheter having a thermal sensor on a distal section thereof; a system controller coupled to the thermal sensor; and
a display configured to graphically display thermography data from the thermal sensor in an annular ring on a screen of the display that is divided into circumferential sections.

20. The thermography system of claim 19 wherein the display comprises a graphic user interface.

21. A thermography instrument, comprising:
a system controller coupled to a thermography data input; and
a display configured to graphically display thermography data from the thermography data input.

22. The thermography instrument of claim 21 wherein the display comprises a graphic user interface.

23. The thermography instrument of claim 21 wherein the system controller is coupled to a plurality of thermography data inputs and wherein the display is configured to display thermography data in a series of concentric rings divided into circumferential sections with each circumferential section correlating to a distinct thermography data input and with adjacent rings representing different thermography data points.

24. The thermography instrument of claim 23 wherein thermography data is displayed such that each circumferential section has a color which is a function of the thermography data from each thermography data input.

25. The thermography instrument of claim 23 wherein difference between blood temperature and the temperature of tissue adjacent the blood is displayed.

26. The thermography system of claim 23 wherein blood temperature is displayed in a center portion of the display.

27. The thermography instrument of claim 21 wherein the thermography data is displayed in an annular ring on a screen of the display that is divided into circumferential sections.

28. The thermography instrument of claim 27 wherein a difference between blood temperature and temperature of tissue adjacent the blood is displayed.

29. The thermography instrument of claim 27 wherein blood temp is displayed in center of display.

30. The thermography system of claim 21 wherein the system controller comprises a CPU.

31. The thermography system of claim 21 wherein the thermal sensor is coupled by a conductor to a connector at a proximal end of the catheter and the connector is coupled to the system controller by an interface cable.

32. The thermography instrument of claim 21 further comprising a pedestal and a keyboard coupled to the display.

33. A thermography instrument, comprising:
a system controller coupled to a thermography data input; and
a display coupled to the system controller configured to graphically display thermography data from at least one data input in a series of concentric rings divided into circumferential sections with each circumferential section correlating to a distinct thermography data input and with adjacent rings representing different thermography data points.

34. The thermography instrument of claim 33 wherein the display comprises a graphic user interface.

35. A thermography instrument, comprising:
a system controller coupled to a plurality of thermography data inputs; and
a display coupled to the system controller and configured to graphically display thermography data from the data inputs in an annular ring on a screen of the display that is divided into circumferential sections, with each circumferential section corresponding to a distinct thermography data input.

36. The thermography instrument of claim 35 wherein the display comprises a graphic user interface.

37. A method of displaying thermography data, comprising:
providing a thermography system, comprising:
a thermography catheter having a thermal sensor on a distal section thereof;
a system controller coupled to the thermal sensor;
a display configured to graphically display thermography data from the thermal sensor;
positioning the thermography catheter in a body of a patient;
detecting thermography data at the thermal sensor; and
graphically displaying the thermography data on display.

38. The method of claim 37 wherein the thermography catheter is positioned within a body vessel of the patient prior to detecting the thermography data.

39. The method of claim 38 wherein the body vessel comprises a coronary artery.

40. The method of claim 37 wherein the display comprises a graphic user interface.

41. The method of claim 37 wherein the thermography catheter comprises a plurality of thermal sensors in a substantially annular array and the display graphically displays the thermography data in a series of concentric rings divided into circumferential sections with each circumferential section correlating to a distinct thermal sensor and with adjacent rings representing different thermography data points.

42. The method of claim 41 wherein temperature data is displayed by the display of a color in each of the circumferential sections and wherein the color is a function of the thermography data from each thermal sensor corresponding to the circumferential section.

43. The method of claim 42 wherein the thermography data displayed comprises a difference between blood temperature and temperature of a body vessel wall adjacent the blood.

44. The method of claim 37 wherein blood temperature is displayed in center of display.

45. The method of claim 37 wherein the thermography data is displayed in an annular ring on a screen of the display that is divided into circumferential sections.

46. The method of claim 45 wherein the thermography data displayed in each circumferential section comprises a difference between blood temperature and temperature of a body vessel wall adjacent the blood.

47. The method of claim 45 wherein blood temperature is displayed in center portion of the annular ring.

48. The method claim 37 wherein the system controller comprises a CPU that converts thermography data from the thermal sensor to graphical data that is communicated to the display.

49. The method of claim 37 wherein the thermal sensor is coupled by a conductor to a connector at a proximal end of the catheter and the connector is coupled to the system controller by an interface cable.

50. The method of claim 41 wherein detecting thermography data comprises detecting temperature data from thermal sensors at a plurality of axial points while the catheter is being axially translated within a body vessel and each concentric ring of the display corresponds to a distinct axial position within the body vessel.

51. The method of claim 50 wherein the thermography system further comprises a mechanical pull back device coupled to a proximal portion of the catheter and the catheter is axially translated by the mechanical pull back device.

52. The method of claim 51 wherein the thermography system further comprises an axial position encoder coupled to the mechanical pull back device and coupled to the system controller and wherein position data of the catheter is communicated from the encoder to the system controller during the axial translation of the catheter.

53. A method of displaying thermography data, comprising:
providing a thermography instrument, comprising:
a system controller coupled to a thermography data input; and
a display configured to graphically display thermography data from the thermography data input;
detecting thermography data from the thermography data input; and
graphically displaying the thermography data on the display.

54. The method of claim 53 wherein the display comprises a graphic user interface.

55. The method of claim 53 wherein the system controller is coupled to a plurality of thermography data inputs and thermography data from the inputs is displayed in a series of concentric rings divided into circumferential sections with each circumferential section correlating to a distinct thermography data input and with adjacent rings representing different thermography data points.

56. The method of claim 55 wherein thermography data is displayed as a color that is a function of the thermography data from each thermography data input for each circumferential section.

57. The method of claim 53 wherein the system controller is coupled to a plurality of thermography data inputs and thermography data from the inputs is displayed in an annular ring on a screen of the display that is divided into circumferential sections.

58. The method of claim 57 wherein thermography data is displayed as a color that is a function of the thermography data from each thermography data input for each circumferential section.

59. The method claim 53 wherein the system controller comprises a CPU that converts thermography data from the thermography data input to graphical data that is communicated to the display.

60. An apparatus for measuring the thermal characteristics of blood vessel in vivo, comprising:
a catheter having a proximal end, a distal end, and a distal section;
an expandable slotted body located at the distal section of said catheter, said expandable slotted body comprising one or more slotted body arms;
one or more vessel wall temperature sensors positioned on said one or more slotted body arms, said one or more vessel wall temperature sensors configured to make contact with a vessel wall when said expandable slotted body is in an expanded state; and
one or more blood temperature sensors positioned on said one or more slotted body arms in a configuration which prevents contact between said one or more blood temperature sensors and said vessel wall when said expandable slotted body is in an expanded state.

61. A thermography catheter having an expandable member disposed on a distal section thereof and a plurality of thermal sensors with a first thermal sensor configured to be at an outer most radial position from a longitudinal axis of the distal section of the catheter with the expandable member in an expanded state and a second thermal sensor configured to disposed radially inward of the first thermal sensor with the expandable member in an expanded state.

62. A thermography method comprising measuring the temperature of a body vessel wall and the temperature of blood adjacent to the body vessel wall contemporaneously.

63. A thermography method, comprising:
providing a catheter having an expandable member disposed on a distal section thereof and a plurality of thermal sensors on a distal section thereof; positioning the distal section of the catheter in a body vessel;
detecting the temperature of a site on the body vessel; and
detecting the temperature of blood adjacent the site of the body vessel contemporaneously with the detection of the temperature of the site on the body vessel.

64. The method of claim 63 further comprising displaying the temperature of the body vessel site and the temperature of the blood on a graphical display.

65. The method of claim 64 further comprising calculating the difference between the blood temperature and the body vessel wall temperature and displaying the result in a graphical color display wherein the color is a function of the temperature difference.

66. A thermography instrument for displaying thermography data taken from a site within a patient, comprising:
a system controller coupled to a thermography data input; and
a display coupled to the system controller and configured to graphically display thermography data from the thermography data input on a graph having thermography data from the thermography data input on a first axis and an axial position of a site from which the thermography data was taken on a second axis.

67. The thermography instrument of claim 66 wherein the display comprises a graphic user interface.

68. The thermography instrument of claim 66 wherein the system controller comprises a plurality of thermography data inputs and thermography data from each of the thermography data inputs is displayed in a graph having a different color from graphs of thermography data taken from other thermography data inputs.

69. The thermography instrument of claim 66 wherein a difference between blood temperature and the temperature of tissue adjacent the blood is displayed on the first axis.
